(19) United States
(12) Patent Application Publication
(10) Pub. No.: US 2022/0351367 A1
(43) Pub. Date: Nov. 3, 2022

(54) CONTINUOUS UPDATE OF HYBRID MODELS FOR MULTIPLE TASKS LEARNING FROM MEDICAL IMAGES

(71) Applicant: AVICENNA.AI, Marseille (FR)

(72) Inventor: Cyril DI GRANDI, La Ciotat (FR)

(73) Assignee: AVICENNA.AI, Marseille (FR)

(21) Appl. No.: 17/319,312

(22) Filed: May 13, 2021

Related U.S. Application Data
(63) Continuation of application No. 17/245,049, filed on Apr. 30, 2021, now abandoned.

(51) Int. Cl. G06T 7/00; G06N 3/08

(52) U.S. Cl. CPC G06T 7/0012; G06N 3/088; G06T 2207/20081; G06T 2207/20084

(57) ABSTRACT

A machine learning method for medical image diagnostic tasks includes (i) iteratively performing an unsupervised learning step of a plurality of unsupervised learning models from a set of unlabeled patient data to extract features from said unlabeled patient data, and (ii) performing on an ad hoc basis a supervised learning step using extracted features to learn a plurality of supervised learning models from a first set of labeled medical images for a first medical image diagnostic task, and from a second set of labeled medical images for a second medical image diagnostic task different from the first medical image diagnostic task.

### CROSS-REFERENCE TO RELATED APPLICATION

This application is a continuation of U.S. patent application Ser. No. 17/245,049 filed Apr. 30, 2021, the entire content of which is incorporated herein by reference.

### FIELD OF THE INVENTION

The present invention relates generally to machine learning methods and systems, and more particularly to machine learning solutions for medical image diagnostic tasks.

### BACKGROUND OF THE INVENTION

Machine learning is making significant progress in clinical decision making and diagnosis. By leveraging available medical images, machine learning techniques attempt to learn hidden patterns and structures in medical images to build models that associate a set of features with the detection of the presence/absence of symptoms, diseases or anomalies.

In this regard, various learning techniques exist that can be roughly classified into supervised and unsupervised approaches.

In supervised approaches, the model is established based on a training set of medical images with the correct responses (i.e. labeled medical images). The bigger the training set of labeled medical images is, the better the constructed model performs. When deployed, the supervised model determines which of the predefined labels is associated with the extracted features from an input medical image. Such supervised models are known to be relatively fast and accurate to determine whether a medical image is malignant or benign.

However, a major downside of the supervised approach is that it relies on labeled medical images, which are not easily available in practice. Labeled medical images can be extremely difficult, even impossible (for rare diseases), to obtain. Furthermore, analysis and annotation of medical images by experts is time and cost consuming. Thus, if there are any, they are usually in very small quantities. In fact, supervised machine learning methods are, in practice, limited to the rare situations where a sufficient amount of labeled medical images is available. Moreover, whatever the size of the training set of labeled medical images, it is difficult to allege that this training set covers all possible malignant cases. New diseases are constantly being discovered.

Some of the above drawbacks can be addressed by unsupervised learning models where the prior knowledge of labels is not required. In this approach, the labeling is performed by the model itself by identifying similarities between the inputs grouped into patterns without involving target attributes.

It is also known to combine the above two approaches to generate, by an unsupervised learning model, referential labels provided as input to a supervised learning model.

Nevertheless, such hybrid machine learning approaches are limited to a specific task objective. The whole learning process should be repeated as many times as envisaged medical image diagnostic tasks. Furthermore, the performance of machine learning models is usually subject to biases in input data, specifically when deployed in a plurality of hospital sites. In addition to introduced medical imaging equipment noise, hidden factors in medical images such as physiological, environmental influence (environmental exposures, such as smoking, sunbathing, or weather conditions), genetic, gender, age, or ethnicity factors may cause performance degradation or, more generally, a performance drift of the deployed unsupervised and/or supervised learning models. Therefore, a continuous end-to-end training and update of these models is usually required.

Nonetheless, continuous update of an ensemble of distributed models is challenging in more than one respect. A first problem may arise with regards to privacy and sensitive information concerns while sharing training sets of labeled medical images. A second problem is that an incremental fine tuning of a medical image learning model generally leads to an accumulation of patches that makes it more and more complicated and cumbersome. Because of constant maintenance and tuning to support various performance drift, models become overly complex (to maintain and evolve). As a third problem and despite a continuous fine tuning, the performance of a medical image learning model will ultimately, sooner or later, reach a plateau (a substantially fixed level) without any significant progress or gain in performance. This requires a paradigm change (e.g. model network topology, data pre- or post-processing, model family change), probably canceling all the previous gains (for example, online training without access to historical data used for the fine-tuning or impossibility to transfer the learned weights to the new model architecture).

### SUMMARY

Various embodiments are directed to addressing the effects of one or more of the problems set forth above. The following presents a simplified summary of embodiments in order to provide a basic understanding of some aspects of the various embodiments. This summary is not an exhaustive overview of these various embodiments. It is not intended to identify key or critical elements or to delineate the scope of these various embodiments. Its sole purpose is to present some concepts in a simplified form as a prelude to the more detailed description that is discussed later.

Some embodiments overcome one or more drawbacks of the prior art, by providing a common backbone/framework for multiple tasks learning from patient data.

Some embodiments provide strategies for distributed monitoring of medical image learning models.

Some embodiments provide long-term deployment of multiple medical image diagnostic tasks.

Some embodiments provide a continuous update of hybrid models for fast multiple tasks learning from medical images.

Some embodiments provide pragmatic machine learning approaches for multiple tasks of medical image classification, segmentation, or regression.

Various embodiments relate to a machine learning system for medical image diagnostic tasks, comprising:
an unsupervised learning module configured to iteratively learn a plurality of unsupervised learning models from a set of unlabeled patient data to extract features from said patient data;
a supervised learning module configured to use the extracted features to learn on an ad hoc basis a plurality of supervised learning models from a first set of labeled medical images for a first medical image diagnostic task, and from a second set of labeled medical images for a second medical image diagnostic task different from the first task.

In accordance with a broad aspect, the unlabeled patient data include unlabeled medical images.

In accordance with another broad aspect, the unsupervised learning module is configured to iteratively learn said plurality of unsupervised learning models independently of the ad hoc supervised learning.

In accordance with another broad aspect, the set of unlabeled medical images is drastically larger than the first and second set of labeled medical images.

In accordance with another broad aspect, the first medical image diagnostic task is a classification task, the second task being a regression task.

In accordance with another broad aspect, the plurality of unsupervised learning models includes a self-supervised learning model and a deep clustering model.

In accordance with another broad aspect, the set of unlabeled patient data comprises a plurality of disjoint sets of unlabeled patient data.

In accordance with another broad aspect, each supervised learning model of said plurality of supervised learning models is intended to be used in one of a plurality of states including a first state wherein an output data of said each supervised learning model is included in computing a result of the first medical image diagnostic task and a second state wherein the output data of said each supervised learning model is excluded from computing the result of the first medical image diagnostic task, the machine learning system further including a supervision module configured to change, based on output data of each supervised learning model being used in the first state and of each supervised learning model being used in the second state, the state of a supervised learning model from one state to another of said plurality of states.

In accordance with another broad aspect, the machine learning system further includes a computing unit configured to combine the output data of supervised learning models being used in the first state to compute the result of the first medical image diagnostic task.

In accordance with another broad aspect, the supervision module is further configured to track performance of each supervised learning model being used in the first state and performance of each supervised learning model being used in the second state.

In accordance with another broad aspect, the supervision module is further configured to change the state of a supervised learning model from one state to another of said plurality of states in response to a remote request.

Various embodiments further relate to a machine learning method for medical image diagnostic tasks, comprising
iteratively performing an unsupervised learning step of a plurality of unsupervised learning models from a set of unlabeled patient data to extract features from said unlabeled patient data;
performing on an ad hoc basis a supervised learning step using extracted features to learn a plurality of supervised learning models from a first set of labeled medical images for a first medical image diagnostic task, and from a second set of labeled medical images for a second medical image diagnostic task different from the first task.

In accordance with a broad aspect, the unlabeled patient data include unlabeled medical images.

In accordance with another broad aspect, the unsupervised learning step is iteratively performed independently of the ad hoc supervised learning step.

In accordance with another broad aspect, the set of unlabeled medical images is drastically larger than the first and second set of labeled medical images.

In accordance with another broad aspect, the first task is a classification task, the second task being a regression task.

In accordance with another broad aspect, the plurality of unsupervised learning models includes a self-supervised learning model and a deep clustering model.

In accordance with another broad aspect, each supervised learning model of said plurality of supervised learning models is intended to be used in one of a plurality of states including a first state wherein an output data of said each supervised learning model is included in computing a result of the first medical image diagnostic task and a second state wherein the output data of said each supervised learning model is excluded from computing the result of the first medical image diagnostic task, the machine learning method further including a first step to change, based on output data of each supervised learning model being used in the first state and of each supervised learning model being used in the second state, the state of a supervised learning model from one state to another of said plurality of states.

In accordance with another broad aspect, the machine learning method further includes a combination step of the output data of supervised learning models being used in the first state to compute the result of the first task.

In accordance with another broad aspect, the machine learning method further includes a tracking step of performance of each supervised learning model being used in the first state and of performance of each supervised learning model being used in the second state.

In accordance with another broad aspect, the machine learning method further includes a second change step of the state of a supervised learning model from one state to another of said plurality of states in response to a remote request.

While the various embodiments are susceptible to various modification and alternative forms, specific embodiments thereof have been shown by way of example in the drawings. It should be understood, however, that the description herein of specific embodiments is not intended to limit the various embodiments to the particular forms disclosed.

It may of course be appreciated that in the development of any such actual embodiments, implementation-specific decisions should be made to achieve the developer's specific goal, such as compliance with system-related and business-related constraints. It will be appreciated that such a development effort might be time consuming but may nevertheless be a routine understanding for those or ordinary skill in the art having the benefit of this disclosure.

### DESCRIPTION OF THE DRAWING

The objects, advantages and other features of the present invention will become more apparent from the following disclosure and claims. The following non-restrictive description of preferred embodiments is given for the purpose of exemplification only with reference to the accompanying drawing in which

FIG. 1 schematically illustrates components of a machine learning system for multiple medical image diagnostic tasks according to various embodiments;

FIG. 2 schematically illustrates process steps of a method for multiple medical image diagnostic tasks according to various embodiments;

FIG. 3 schematically illustrates elements of a computing device according to various embodiments;

FIG. 4 schematically illustrates a continuous supervision and training workflow of unsupervised and supervised leaning models according to various embodiments.

### DETAILED DESCRIPTION OF ILLUSTRATIVE EMBODIMENTS

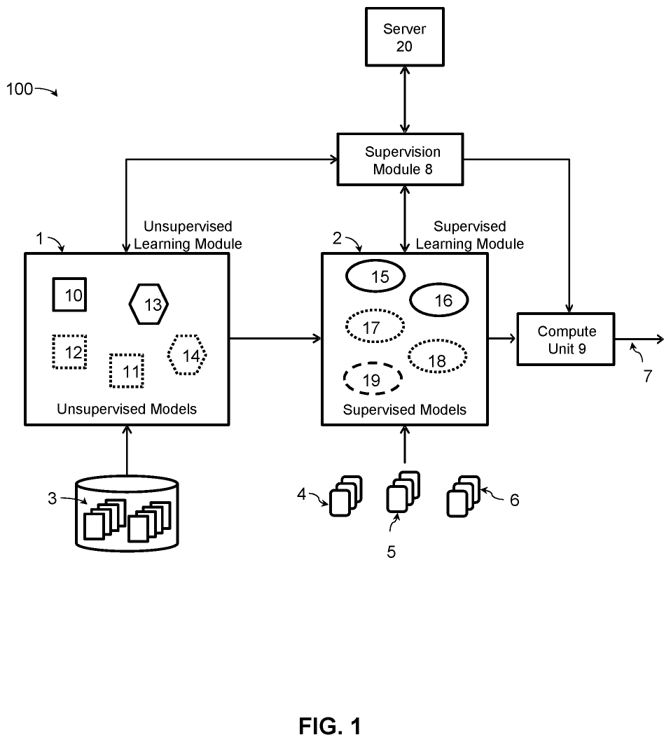

With reference to FIG. 1, there is shown a machine learning system 100 for medical image diagnostic tasks. Medical images may be ultrasound image, magnetic resonance image, radiography image, scan image, 3D medical image or, more generally, any digital or capable of being digitized medical image. Medical image-based diagnostic tasks comprise classification, segmentation and/or regression tasks. For instance, these tasks includes intracranial hemorrhage detection, diagnosis of different types and stages of cancer (such as breast cancer or colon cancer), neurological disease detection, fractures or musculoskeletal injury detection, cardiovascular abnormality identification, or detection of thoracic complications.

The machine learning system 100 includes an unsupervised learning (or pre-training) module 1. This unsupervised learning module 1 is configured to perform an unsupervised learning from a set of unlabeled (or label-free) medical images 3. The unsupervised learning module 1 is intended to find out latent patterns/structures in unlabeled medical images 3 and extract therefrom general features suitable for multiple tasks learning. To this end, the unsupervised learning module 1 iteratively (i.e. continuously, recurrently, repeatedly, or in an iterative manner) learns a plurality of unsupervised learning models 10-14 from unlabeled medical images 3. Indeed, unlabeled medical images 3 are iteratively collected (or amassed) and an unsupervised learning of the plurality of unsupervised learning models 10-14 is repeatedly performed therefrom. Accordingly, the unsupervised learning module 1 continuously accumulates knowledge about continuously collected unlabeled medical images 3.

The plurality of unsupervised learning models 10-14 is configured to extract features from the set of unlabeled medical images 3.

In one embodiment, the unsupervised learning models 10-14 may include a self-supervised model and/or a deep clustering model. The unlabeled medical images 3 are provided as input to the self-supervised model whose output is communicated to the deep clustering model. The self-supervised model is, for instance, a self-organizing neural network map (such as Kohonen self-organizing neural network). The deep clustering model is any unsupervised classification algorithm able to identify inherent features within the self-supervised model output. The deep clustering model is, for instance, fuzzy C-means (FCM) clustering, K-means (KM) clustering, iterative self-organizing data analysis technique algorithm (ISODATA), unsupervised neural networks, or expectation-maximization (EM) algorithm. The deep clustering model is configured to group output of the self-supervised model into a pre-defined or an automatically determined number of features. For this purpose, the deep clustering model minimizes an intra-cluster variation or, more generally, optimizes a cost function involving distances between output of the self-supervised model and cluster prototypes. Advantageously, the deep clustering model summarizes general and meaningful features of unlabeled medical images 3 for multiple tasks.

The machine learning system 100 further includes a supervised learning module 2. This supervised learning module 2 is configured to use the extracted features to learn on an ad hoc basis (i.e. for a particular purpose or need) a plurality of supervised learning models 15-19 from different sets of labeled medical images 4-6 for learning different tasks. The extracted features from the unlabeled medical images 3 by the unsupervised learning module 1 are used as initial or referential labels by the supervised learning models 15-19 for learning multiple tasks represented by corresponding sets of labeled medical images 4-6. Each set of labeled medical images 4-6 is associated with a predefined classification, segmentation or regression task.

In other words, from time to time a supervised fine-tuning is performed for a specific purpose (i.e. on an ad hoc or occasionally) by the supervised learning models 15-19 by using automatically extracted features from unlabeled medical images 3 to learn multiple tasks from associated sets of labeled medical images 4-6. Thus, the unsupervised learning module 1 automatically and iteratively creates, from unlabeled medical images 3, labels that can be subsequently used on an ad hoc basis as starting points for the supervised learning models 15-19 to learn multiple tasks from associated sets of labeled medical images 4-6. Advantageously, by providing specific sets of labeled medical images 4-6, a plurality of diagnostic tasks may be handled by the machine learning system 100.

In one embodiment, the supervised learning module 2 includes a supervised learning model 15 intended to learn from a specific set of labeled medical images 4. Accordingly, a first and a second supervised learning model 15, 16 may be configured to learn, respectively, from a first set of labeled medical images 4 and from a second set of labeled medical image 5 different from the first one.

Advantageously, the supervised fine-tuning performed by the supervised learning module 2 requires much less data than the same task without pre-training performed by the unsupervised learning module 1. Therefore, the set of unlabeled medical images 3 is drastically (i.e. significantly or considerably) larger than each of the sets of labeled medical images 4-6. In other words, each of the provided sets of labeled medical images 4-6 is very small compared to the set of unlabeled medical images 3 (for instance, tens of medical images in each set of labeled medicals images 4-6 versus tens of thousands or unbounded number of unlabeled medical images 3). In one embodiment, the unlabeled medical images 3 are provided by a Picture Archiving and Communication Systems (PACS) or, more generally, a medical image database system continuously expanded with new unlabeled medical images of a hospital site. In another embodiment, the set of unlabeled medical images 3 comprises a plurality of disjoint sets of unlabeled medical images (for example, on the premises of different data providers). In practice, unlabeled medical images 3 are, in comparison to labeled medical images 4-6, more abundant. Collecting large data-sets of unlabeled medical images 3 is much easier and less costly than preparing labeled medical images 4-6. Accordingly, an intense medical images analysis by experts can be avoided (no massive expert labeling is required).

More generally, a plurality of medical image diagnostic tasks is achieved through a two-step hybrid approach. On one hand, a continuous or recurrent unsupervised pre-training for global meaningful features extraction from a large set of unlabeled medical images 3 is maintained. On the other hand, an ad hoc or occasional supervised fine-tuning using extracted features and a plurality of small sets of labeled medical images 4-6 associated to predefined different diagnostic tasks is performed.

Advantageously, a large number of different tasks defined by different sets of labeled medical images 4-6 can be performed by fine-tuning a same pre-trained unsupervised learning model 10-14. It results in a unique common backbone or framework enabling multiple tasks learning.

The unsupervised learning module 1 is configured to iteratively learn the plurality of unsupervised learning models 10-14 independently of the ad hoc supervised learning from labeled medical images 4-6 performed by the supervised learning module 2. In other words, the unsupervised learning and the supervised learning, respectively, performed by the unsupervised learning module 1 and the supervised learning module 2 are independent or decorrelated. Advantageously, this decorrelation avoids starting from scratch when the machine learning system 100 handles a new medical image diagnostic task and/or is provided with a new set of labeled medical images 4-6. In view of this decorrelation, the unsupervised learning module 1 and the supervised learning module 2 may be marketed in combination or separately. The unsupervised learning is performed recurrently or regularly online or offline, while the supervised learning is performed on-demand or on ad-hoc basis. The supervised learning may be performed for a new medical image diagnostic task or for an amended set of labeled medical images 4-6 for a specific task.

Advantageously, the continuous unsupervised learning from unlabeled medical images 3 improves the relevance of extracted features for multiple tasks (the larger the collected set of unlabeled medical images 3 is, the more relevant the extracted features are for various tasks). Moreover, an ad hoc supervised learning reduces the need for hard-to-get labeled medical images 4-6.

In one embodiment, the same set of unlabeled medical images 3 is used to generate any number of pre-trained models by changing the unsupervised learning models 10-14, thus further increasing the number of possible medical image diagnostic tasks to be learned by the supervised learning module 2.

In order to improve performance of the machine learning system 100 with respect to medical images biases, a plurality of supervised and/or unsupervised learning models 10-19 is considered. In fact, unlabeled as well as labeled medical images 3-6 may be differently biased from one deployment site to another of the machine learning system 100 (because of site-specific hidden factors, implicit features of medical site patients, or specific medical image equipment).

The plurality of supervised learning models 15-19 (respectively, unsupervised learning models 10-14) may comprise different versions, revisions or editions of a same model or of different models, differently tuned copies of a same model, alternative or equivalent embodiments or implementations of a same model or of different models, or more generally a plurality of models able to perform a same function.

Advantageously, the use of a plurality of supervised learning models 15-19 (respectively, unsupervised learning models 10-14) allows to cope with differently biased medical images.

In one embodiment, each of the supervised/unsupervised learning models 10-19 is intended to be used in one of a plurality of states (mode or stage of its lifecycle) including
an online state (i.e. active or functioning state) wherein it receives input data and its output data is included by the machine learning system 100 in computing the task result 7 (the prediction), i.e. it contributes at the level of the unsupervised learning module 1 or at the level of the supervised learning module 2 to the prediction. That is to say, its output data is taken into account in the calculation of the task result 7;
a quarantined state (i.e. under review or available as replacement state) wherein it receives, like in the online state, input data but its output data is excluded from computing the task result 7, i.e. it does not contribute to the prediction. That is to say, its output data is not taken into account in the calculation of the task result 7.

An unsupervised or supervised learning model 10-19 of the machine learning system 100 may be further intended to be used
in an archived state wherein it neither receives input data nor generates output data; or
in a retired state wherein it is scheduled for removal from the machine learning system 100 (for example, after a legal retention period).

Each time the unsupervised learning module 1 or the supervised learning module 2 receives an input medical image, respectively, from the unlabeled medical images 3 or from a set of labeled medical images 4-6 associated with a predefined task, the supervised and unsupervised learning models 10-19 which are in the online state (shown in solid line in FIG. 1) and in the quarantined state (shown in dotted line in FIG. 1) are launched and generate their respective output data.

In one embodiment, the state of a supervised learning model 15-19 is defined per medical image diagnostic task (i.e. per set of labeled medical images 4-6). In other words, a supervised learning model 15-19 may be in first state when learning from a first set of labeled medical images 4 and in a second state when learning from a second set of labeled medical images 5.

In one embodiment, the machine learning system 100 further includes a computing unit 9 configured to combine the individual output of supervised learning models 15-19 being used in the online state to compute a task result 7 (the prediction). The computing unit 9 is configured to compute, based on output data generated by supervised learning models 15-19 being used in the online state, the task result 7.

In one embodiment, the task result 7 is computed by majority vote between the output data of supervised learning models 15-19 being used in the online state. In another embodiment, the task result 7 is a weighted average of output data of supervised learning models 15-19 being used in the online state. In another embodiment, the task result 7 computation is based on a bayesian algorithm, a multi-armed bandit algorithm, heuristics, or any other logic model framework applied to the individual output data of the supervised learning models 15-19 being used in the online state.

More generally, the task result 7 is based on a combination of output data generated by supervised learning models 15-19 being used in the online state. Advantageously, a plurality of supervised learning models 15-19 with potentially different biases perform collectively better than individually for task result 7 prediction.

The machine learning system 100 includes a supervision module 8 configured to monitor the unsupervised learning module 1 and the supervised learning module 2. The supervision module 8 dynamically manages the states of the unsupervised and supervised learning models 10-19.

The supervision module 8 is able to change the state of an unsupervised or supervised learning model 10-19 being used in the online state or in the quarantined state from one state to another based on its output data. In this regards, the supervision module 8 collects the individual output data of unsupervised and supervised learning models 10-19 being used in the online state and in the quarantined state. Based on collected output data, the supervision module 8 decides which unsupervised and supervised learning models 10-19 can contribute, or not, to the computation of the task result 7 (the prediction) and changes its state accordingly.

More generally, the supervision module 8 is configured to track performance of unsupervised and supervised learning models 10-19 being used in the online state and those being used in the quarantined state. In one embodiment, the supervision module 8 is provided with predefined rules (or policies) to be applied on historical performance (performance track record) over a predefined sliding window to determine state changes of a deployed unsupervised/supervised learning models 10-19 being used in the online state or in the quarantined state.

By changing the state of one or more of the unsupervised/supervised learning models 10-15, those contributing to the task result 7 may be modified to improve task learning. Based on predefined rules, the supervision module 8 detects performance improvement or degradation and changes accordingly the states of deployed models. These rules are for instance:
change the use state of a deployed model from “online” to “quarantined” or from “quarantined” to “archived” when its performance according to a first metric falls below a predefined threshold;
change the use state of a deployed model from “quarantined” to “online” when its performance according to a second metric is above a predefined threshold after some duration (a predefined sliding window or number of runs for example).

In one embodiment, the first metric and/or the second metric are a predefined statistical value (such as, an average, a maximum, a minimum, a median, or a standard deviation) of output data generated by a deployed model over a predefined duration such as a sliding window like a weekly, a monthly or a quarterly moving average, or of a predefined number of output data generated by the deployed model such as the fifty, or the hundred most recent output data.

The collection of individual output data of unsupervised and supervised learning models 10-19 being used in the online state and in the quarantined state, advantageously, allows the machine learning system 100 to continuously have its own evaluation metrics (e.g. Area Under the Curve of Receiver Characteristic Operator which can be evaluated over time and introduced into a decision strategy) for checking the performance of these deployed supervised and unsupervised learning models 10-19.

In one embodiment, based on collected individual output data of supervised learning models 15-19 being used in the online state, the supervision module 8 may decide on how a supervised learning model 15-19 being used in the online mode contributes (weights) in the computation of the task result 7. For example, an average performance (i.e. accuracy) over a predefined sliding window or over a predefined number of most recent runs may be used as weights for computing a weighted average of supervised learning model 15-19 output data by the computing unit 9. Accordingly, a performance track record of supervised learning models 15-19 being used in the online state is provided by the supervision module 8 to the computing unit 9 to compute the task result 7.

More generally, based on historical performance of unsupervised and/or supervised learning models 10-19 over a predefined sliding window, the supervision module 8 and the computing unit 9 contribute, advantageously, to a continuous local monitoring and update of the machine learning system 100. In fact, a sliding window of historical performance including a batch of recent runs may be used by the supervision module 8 to assess performance of each unsupervised/supervised learning model 10-19 and to select the most relevant ones to contribute to the prediction by changing their state to online state.

In one embodiment, the supervision module 8 changes the state of an unsupervised or supervised learning model 10-19 in response to a remote request (such as archiving, retiring, change to online or to quarantined state of a certain model).

Advantageously, the dynamic addition of new models in the unsupervised or supervised learning modules 1, 2 and the step-by-step upgrade or retirement of existing ones by changing their use states (e.g. an observation period in the quarantined state before an upgrade to online state or before a downgrade to archived state) allow efficient and smooth change without loss of previous results and allow to maintain long term performance.

In one embodiment, a Multi Decision Criteria Analysis (MDCA) and/or Operations Research (OR) scheme is used for producing, from the collected individual output data of deployed unsupervised and/or supervised learning models 10-19, optimal rules respectively, for the supervision module 8 and for the computing unit 9.

Advantageously, the unsupervised learning may be performed online or offline and continuously updated by the supervision module 8, independently of the future medical image diagnostic tasks.

In one embodiment, a remote server 20 is configured to monitor the on-site supervision module 8. This remote server 20 is, in one embodiment, external to the local network of the deployment site where the machine learning system 100 is deployed. When a plurality of machine learning systems 100 are deployed in a plurality of sites, the remote server 20 is able to acquire knowledge on their performances which can be used to define a global strategy for updating unsupervised/supervised learning models 10-19 in each machine learning system.

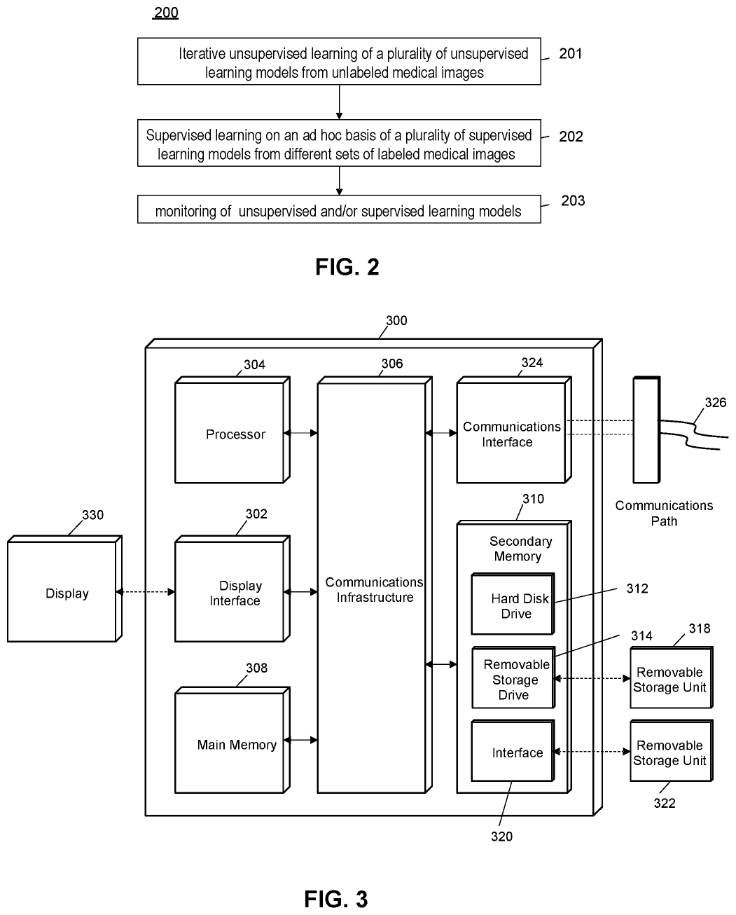

With reference to FIG. 2, there are shown process steps of a machine learning method 200 for medical image diagnostic tasks. This method 200 includes an iterative unsupervised learning step 201 of a plurality of unsupervised learning models from the unlabeled medical images 3 to extract therefrom meaningful features for multiple tasks. These features are subsequently used by a supervised learning step 202 for learning on an ad hoc basis a plurality of supervised learning models from different sets of labeled medical images 4-6 for different medical image diagnostic tasks.

In one embodiment, the iterative unsupervised learning step 201 comprises a learning step of a self-supervised model and a deep clustering step. The deep clustering step groups the output of the self-supervised model into a pre-defined or an automatically determined number of features used by the supervised learning step 202.

In one embodiment, each unsupervised or supervised learning model 10-19 is intended to be used in one of a plurality of states as described above (online, quarantined, archived, or retired for example). During a monitoring step 203, the state of an unsupervised or supervised learning model may be changed by the supervision module 8 from one state to another based on performance of models being used in the online state and quarantined state. This monitoring step 203 includes tracking performance of unsupervised and supervised learning models 10-19 being used in the online state and in the quarantined state. By periodically adapting the state (online, quarantined, archived, or retired for example) of the deployed unsupervised/supervised leaning models 10-19 based on their performance tracking, the monitoring step 201, advantageously, ensures task performance above a predefined lower limit corresponding to a confidence level, thus providing performance stability. Indeed, the use state of deployed unsupervised/supervised learning models 10-19 may be automatically changed in a proactive manner as soon as the task performance falls below the predefined lower limit.

The machine learning method 200 improves generalization (i.e. improve robustness on unseen data) of the learned tasks because it does not require the addition of new labeled medical images 4-6 as is generally the case for end-to-end supervised training.

Advantageously, the above described hybrid approach for learning multiple clinical tasks from medical images combines the advantages of unsupervised learning (no labeling required) to extract meaningful features for multiple tasks which are fed as a starting point to a supervised fine-tuning (fast and accurate). Accordingly, high performance with very few labeled medical images 4-6 may be achieved. By combining an iterative unsupervised learning step with an ad hoc supervised fine-tuning of a given task, the advantages of unsupervised learning (no labeling required) to extract meaningful features is used as a starting point for at least a supervised specific model using medical images with adequate labels, but in a much smaller quantity.

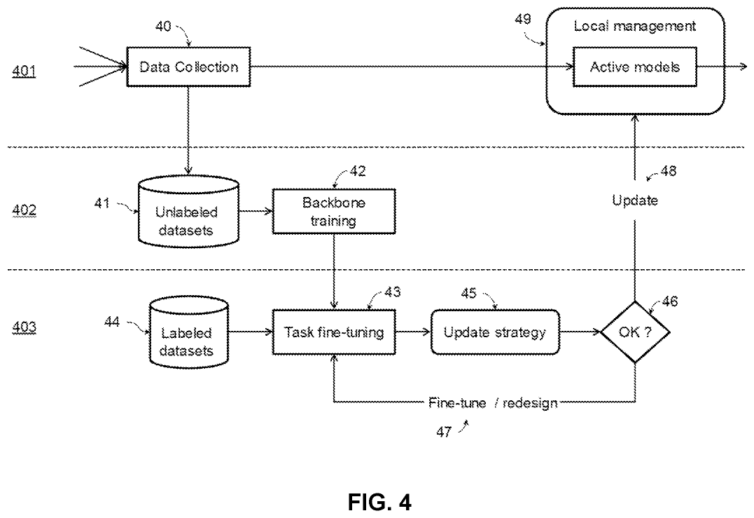

FIG. 4 shows continuous supervision and training workflow of unsupervised and supervised leaning models 10-19. During a prospective collection phase 401, new unlabeled medical images are continuously collected (step 40) from diverse sources (for instance, various PACS). These newly collected unlabeled medical images are provided to unlabeled datasets 42 so as to iteratively/repeatedly update (step 41) backbone models (i.e. the unsupervised learning models 10-14) during a generic pre-training phase 402. During a task specific phase 403, updated unsupervised learning models 10-14 outputs are used to fine-tune on an ad hoc basis a given task (step 43) which are performed by supervised learning models 15-19 based on corresponding labeled dataset 44 of medical images. Based on the comparison of a task result with a predefined threshold (test 46), an update strategy 45 actively monitors the task performance. For instance, a fine-tuning or a redesign (step 47) of deployed supervised learning models 15-19 may be considered by changing the use state (online, quarantined, and archived, for example) of at least one of these models. Also, an update 48 of the deployed models' performance on labeled medical images may be communicated to a local management unit 49 configured to track performance of local active unsupervised learning models (those being used in the online or quarantined state). Provided with task performance and locally collected unlabeled medical images, the local management unit 49 is able to manage locally deployed unsupervised/supervised learning models.

Advantageously, the above described machine learning method and system are privacy-preserving by design, removing an important barrier in health care applications, because sharing unlabeled medical images is less privacy invasive than sharing labeled ones.

Advantageously, given the small size of labeled medical images sets allowed by the present disclosure,
the supervised learning step may be performed in real time or near-real time;
the machine learning system may provide a large number of specific medical image diagnostic tasks;
the development cost of such tasks remains competitive;
user input data are significantly reduced, thus alleviating the data entry burden for the user while encouraging high annotation quality, as well as reducing the required computing capacity.

It is to be noted that references throughout specification and figures to unlabeled medical images is to be regarded as illustrative instead of limiting because the teachings of this disclosure may, more generally, be applied to unlabeled patient data. The teaching of the present disclosure is not limited to unlabeled patient data represented by images. Instead or in addition to unlabeled medical images, unlabeled patient data may include
quantitative data (or features) such as vital signs (for instance, temperature or blood pressure), personal statistics (for instance, sex, age, body, weight), or blood/microbiology analysis results; and/or
qualitative features such as presence/absence of symptoms, or outcomes/response to a drug (allergies/immunizations).

FIG. 3 illustrates a computer system 300 in which embodiments of the present disclosure, or portions thereof, may be implemented as computer-readable code. For example, one or more (e.g., each) of the unsupervised learning module 1, supervised learning module 2, computing unit 9, supervision module 8, remote server 20, and other device described herein may be implemented in the computer system 300 using hardware, software, firmware, non-transitory computer readable media having instructions stored thereon, or a combination thereof and may be implemented in one or more computer systems or other processing systems. Hardware, software, or any combination thereof may embody modules and components used to implement the method of FIG. 2.

If programmable logic is used, such logic may execute on a commercially available processing platform configured by executable software code to become a specific purpose computer or a special purpose device (e.g., programmable logic array, application-specific integrated circuit, etc.). A person having ordinary skill in the art may appreciate that embodiments of the disclosed subject matter can be practiced with various computer system configurations, including multi-core multiprocessor systems, minicomputers, mainframe computers, computers linked or clustered with distributed functions, as well as pervasive or miniature computers that may be embedded into virtually any device. For instance, at least one processor device and a memory may be used to implement the above-described embodiments.

A processor unit or device as discussed herein may be a single processor, a plurality of processors, or combinations thereof. Processor devices may have one or more processor “cores.” The terms “computer program medium,” “non-transitory computer readable medium,” and “computer usable medium” as discussed herein are used to generally refer to tangible media such as a removable storage unit 318, a removable storage unit 322, and a hard disk installed in hard disk drive 312.

Various embodiments of the present disclosure are described in terms of this example computer system 300. After reading this description, it will become apparent to a person skilled in the relevant art how to implement the present disclosure using other computer systems and/or computer architectures. Although operations may be described as a sequential process, some of the operations may in fact be performed in parallel, concurrently, and/or in a distributed environment, and with program code stored locally or remotely for access by single or multi-processor machines. In addition, in some embodiments the order of operations may be rearranged without departing from the spirit of the disclosed subject matter.

Processor device 304 may be a special purpose or a general purpose processor device specifically configured to perform the functions discussed herein. The processor device 304 may be connected to a communications infrastructure 306, such as a bus, message queue, network, multi-core message-passing scheme, etc. The network may be any network suitable for performing the functions as disclosed herein and may include a local area network (LAN), a wide area network (WAN), a wireless network (e.g., WiFi), a mobile communication network, a satellite network, the Internet, fiber optic, coaxial cable, infrared, radio frequency (RF), or any combination thereof. Other suitable network types and configurations will be apparent to persons having skill in the relevant art. The computer system 300 may also include a main memory 308 (e.g., random access memory, read-only memory, etc.), and may also include a secondary memory 310. The secondary memory 310 may include the hard disk drive 312 and a removable storage drive 314, such as a floppy disk drive, a magnetic tape drive, an optical disk drive, a flash memory, etc.

The removable storage drive 314 may read from and/or write to the removable storage unit 318 in a well-known manner. The removable storage unit 318 may include a removable storage media that may be read by and written to by the removable storage drive 314. For example, if the removable storage drive 314 is a floppy disk drive or universal serial bus port, the removable storage unit 318 may be a floppy disk or portable flash drive, respectively. In one embodiment, the removable storage unit 318 may be non-transitory computer readable recording media.

In some embodiments, the secondary memory 310 may include alternative means for allowing computer programs or other instructions to be loaded into the computer system 300, for example, the removable storage unit 322 and an interface 320. Examples of such means may include a program cartridge and cartridge interface (e.g., as found in video game systems), a removable memory chip (e.g., EEPROM, PROM, etc.) and associated socket, and other removable storage units 322 and interfaces 320 as will be apparent to persons having skill in the relevant art.

Data stored in the computer system 300 (e.g., in the main memory 408 and/or the secondary memory 310) may be stored on any type of suitable computer readable media, such as optical storage (e.g., a compact disc, digital versatile disc, Blu-ray disc, etc.) or magnetic tape storage (e.g., a hard disk drive). The data may be configured in any type of suitable database configuration, such as a relational database, a structured query language (SQL) database, a distributed database, an object database, etc. Suitable configurations and storage types will be apparent to persons having skill in the relevant art.

The computer system 300 may also include a communications interface 324. The communications interface 324 may be configured to allow software and data to be transferred between the computer system 300 and external devices. Exemplary communications interfaces 324 may include a modem, a network interface (e.g., an Ethernet card), a communications port, a PCMCIA slot and card, etc. Software and data transferred via the communications interface 324 may be in the form of signals, which may be electronic, electromagnetic, optical, or other signals as will be apparent to persons having skill in the relevant art. The signals may travel via a communications path 326, which may be configured to carry the signals and may be implemented using wire, cable, fiber optics, a phone line, a cellular phone link, a radio frequency link, etc.

The computer system 300 may further include a display interface 402. The display interface 302 may be configured to allow data to be transferred between the computer system 300 and external display 330. Exemplary display interfaces 302 may include high-definition multimedia interface (HDMI), digital visual interface (DVI), video graphics array (VGA), etc. The display 330 may be any suitable type of display for displaying data transmitted via the display interface 302 of the computer system 300, including a cathode ray tube (CRT) display, liquid crystal display (LCD), light-emitting diode (LED) display, capacitive touch display, thin-film transistor (TFT) display, etc.

Computer program medium and computer usable medium may refer to memories, such as the main memory 308 and secondary memory 310, which may be memory semiconductors (e.g., DRAMs, etc.). These computer program products may be means for providing software to the computer system 300. Computer programs (e.g., computer control logic) may be stored in the main memory 308 and/or the secondary memory 310. Computer programs may also be received via the communications interface 324. Such computer programs, when executed, may enable computer system 300 to implement the present methods as discussed herein. In particular, the computer programs, when executed, may enable processor device 304 to implement the methods illustrated by FIG. 2, as discussed herein. Accordingly, such computer programs may represent controllers of the computer system 300. Where the present disclosure is implemented using software, the software may be stored in a computer program product and loaded into the computer system 300 using the removable storage drive 314, interface 320, and hard disk drive 312, or communications interface 324.

The processor device 304 may comprise one or more modules or engines configured to perform the functions of the computer system 300. Each of the modules or engines may be implemented using hardware and, in some instances, may also utilize software, such as corresponding to program code and/or programs stored in the main memory 308 or secondary memory 310. In such instances, program code may be compiled by the processor device 304 (e.g., by a compiling module or engine) prior to execution by the hardware of the computer system 300. For example, the program code may be source code written in a programming language that is translated into a lower level language, such as assembly language or machine code, for execution by the processor device 304 and/or any additional hardware components of the computer system 300. The process of compiling may include the use of lexical analysis, preprocessing, parsing, semantic analysis, syntax-directed translation, code generation, code optimization, and any other techniques that may be suitable for translation of program code into a lower level language suitable for controlling the computer system 300 to perform the functions disclosed herein. It will be apparent to persons having skill in the relevant art that such processes result in the computer system 300 being a specially configured computer system 300 uniquely programmed to perform the functions discussed above.

1. A machine learning system for medical image diagnostic tasks, comprising:
an unsupervised learning module configured to iteratively learn a plurality of unsupervised learning models from a set of unlabeled patient data to extract features from said unlabeled patient data;
a supervised learning module configured to use the features extracted from the unlabeled data to learn on an ad hoc basis a plurality of supervised learning models from a first set of labeled medical images for a first medical image diagnostic task, and from a second set of labeled medical images for a second medical image diagnostic task different from the first medical image diagnostic task, wherein the learning by the unsupervised learning module and the learning by the supervised learning module are de-correlated from one another.

2. The machine learning system of claim 1, wherein the unlabeled patient data include unlabeled medical images.

3. The machine learning system of claim 1, wherein the unsupervised learning module is configured to iteratively learn said plurality of unsupervised learning models independently of the ad hoc supervised learning.

4. The machine learning method of claim 2, wherein the set of unlabeled medical images is larger than the first and second set of labeled medical images.

5. The machine learning system of claim 1, wherein the first task is a classification task, and the second task is a regression task.

6. The machine learning system of claim 1, wherein the plurality of unsupervised learning models includes a self-supervised learning model and a deep clustering model.

7. The machine learning system of claim 1, wherein the set of unlabeled patient data comprises a plurality of disjoint sets of unlabeled patient data.

8. The machine learning system of claim 1, wherein
each supervised learning model of said plurality of supervised learning models is used in one of a plurality of states including a first state wherein an output data of said supervised learning model is included in computing a result of the first medical image diagnostic task and a second state wherein the output data of said supervised learning model is excluded from computing the result of the first medical image diagnostic task,
the machine learning system further including a supervision module configured to change, based on output data of each supervised learning model being used in the first state and of each supervised learning model being used in the second state, the state of a supervised learning model from one state to another of said plurality of states.

9. The machine learning system of claim 8, further including a processor configured to combine the output data of supervised learning models being used in the first state to compute the result of the first medical image diagnostic task.

10. The machine learning system of claim 8, wherein the supervision module is further configured to track performance of each supervised learning model being used in the first state and performance of each supervised learning model being used in the second state.

11. The machine learning system of claim 8, wherein the supervision module is further configured to change the state of a supervised learning model from one state to another of said plurality of states in response to a remote request.

12. A machine learning method for medical image diagnostic tasks, comprising:
iteratively performing an unsupervised learning step of a plurality of unsupervised learning models from a set of unlabeled patient data to extract features from said unlabeled patient data; and
performing on an ad hoc basis a supervised learning step using the features extracted from unlabeled patient data during the unsupervised step to learn a plurality of supervised learning models from a first set of labeled medical images for a first medical image diagnostic task, and from a second set of labeled medical images for a second medical image diagnostic task different from the first medical image diagnostic task, wherein the unsupervised learning step and the supervised learning step are de-correlated from one another.

13. The machine learning method of claim 12, wherein the unlabeled patient data include unlabeled medical images.

14. The machine learning method of claim 12, wherein the unsupervised learning step is iteratively performed independently of the ad hoc supervised learning step.

15. The machine learning method of claim 13, wherein the set of unlabeled medical images is larger than the first and second set of labeled medical images.

16. The machine learning method of claim 12, wherein the first medical image diagnostic task is a classification task, and the second medical image diagnostic task is a regression task.

17. The machine learning method of claim 12, wherein the plurality of unsupervised learning models includes a self-supervised learning model and a deep clustering model.

18. The machine learning method of claim 12, wherein
each supervised learning model of said plurality of supervised learning models is used in one of a plurality of states including a first state wherein an output data of said supervised learning model is included in computing a result of the first medical image diagnostic task and a second state wherein the output data of said supervised learning model is excluded from computing the result of the first medical image diagnostic task,
the machine learning method further including a first step of changing, based on output data of each supervised learning model being used in the first state and of each supervised learning model being used in the second state, the state of a supervised learning model from one state to another of said plurality of states.

19. The machine learning method of claim 18, further including a step of combining the output data of supervised learning models being used in the first state to compute the result of the first medical image diagnostic task.

20. The machine learning method of claim 18, further including a step of tracking performance of each supervised learning model being used in the first state and performance of each supervised learning model being used in the second state.

21. The machine learning method of claim 18, further including a second step of changing the state of a supervised learning model from one state to another of said plurality of states in response to a remote request.
